(19) United States
(12) Patent Application Publication
(10) Pub. No.: US 2019/0053751 A1
(43) Pub. Date: Feb. 21, 2019

(54) SMART BED

(71) Applicant: Evelyn TORRES, Warsaw, IN (US)

(72) Inventor: Evelyn TORRES, Warsaw, IN (US)

(21) Appl. No.: 16/087,568

(22) PCT Filed: Mar. 22, 2017
(86) PCT No.: PCT/US17/23627

§ 371 Date: Sep. 21, 2018

Related U.S. Application Data
(60) Provisional application No. 62/311,836, filed on Mar. 22, 2016.

(51) Int. Cl. A61B 5/00; A61G 7/057; A61H 23/00; A61B 5/103; A61B 5/01

(52) U.S. Cl. CPC A61B 5/447; A61H 2201/5082; A61H 23/00; A61B 5/1036; A61B 5/6892; A61B 5/01; A61G 2203/34; A61G 2203/46; A61H 2201/0146; A61H 2201/0149; A61H 2201/1654; A61H 2201/501; A61H 2201/5043; A61H 2201/5061; A61H 2201/5071; A61G 7/05784

(57) ABSTRACT

The present disclosure provides a device for use with a bed, chair, or other furniture for seating, reclining, or lying down, which comprises one or more pressure sensors, a perforated air-flow manifold, a plurality of vibrational motors, and a controller unit for the prevention and/or treatment of bed sores in bedridden patients, as well as methods of use thereof.

### RELATED APPLICATIONS

This application claims the benefit of U.S. Provisional Application No. 62/311,836, filed Mar. 22, 2016, the contents of which are incorporated herein by reference in its entirety.

### FIELD OF THE INVENTION

This invention relates to the field of devices, such as mattresses, mattress covers and beds, for the prevention and/or treatment of bed sores.

### BACKGROUND

As Americans live longer, the number of hospitals and nursing homes treating patients for long-term care has increased dramatically. One of the most common problems in the care of long-term bedridden patients is the development of decubitus ulcers or bed sores.

Bed sores are localized areas of cellular necrosis occurring in pressure areas of the patient's body, mainly in bony prominences thereof, and are often caused by constriction of blood flow to capillary vessels due to prolonged localized pressure applied to the body. They result from the pressure exerted on the skin and subcutaneous tissues by the skeletal bony prominences and the object on which the patient rests, such as a bed. The cutaneous tissues are progressively broken down leading to destruction of underlying soft tissue. Once this ulcer forms it is quite painful and very slow to heal. Bacterial infections are difficult to avoid and frequently prolong the healing process. Bedridden patients often feel pressures and pain on their bony prominences even without developing bed sores.

Bedridden patients who are immobilized for a medical cause (e.g., to allow a broken bone to heal) or who are immobile for any reason (e.g., depression, coma, etc.) tend to develop bed sores on body surfaces that are in prolonged contact with bed sheeting or with the covering of any other support surface. Furthermore, the bedridden patient may unconsciously favor resting on body surfaces that have recovering wounds resulting, e.g., from surgery, accident or other trauma. No matter what the cause, when air/oxygen is prevented from reaching the skin of a body part, and when normal perspiration is prevented for a prolonged period, the result is non-optimal healing and possible infection of an existing wound, or, in the case of no wound at the outset, the formation of bed sores. In extreme cases, an undiagnosed, untreated, or inadequately treated bedsore can lead to osteomyelitis, septicemia, and even death.

Numerous efforts have been put forth in the past to devise various cushions, pads, bandages, dressings, mattress modifications, and the like, for attempting to alleviate the pressure on bed sores and thereby promote healing. However, because of the materials used, or the configurations developed, or the expense of making and using the more elaborate devices, there has been no completely satisfactory solution to the problem.

The prevention or treatment of bedsores in bedridden patients has been addressed using many methods, for example: a mattress depressor which can create a depression in a selected area of a mattress in order to relieve pressure around the sore; a bed sore preventing apparatus comprising an air mattress with groups of air cells which, independent of each other, are pneumatically controlled to control pressure distribution; a bed sore prevention device in an invalid bed arrangement, such that the bed may be tilted to shift the weight distribution of the patient from one side to the other side; a bed or mattress surface entirely covered with rows of small rollers for moving a patient to various positions on the mattress; disposable pads which absorb and distribute the pressure of a patient's body and provide lift of the body away from the bed in the area of the bed sore.

Several factors play an important role in the development bed sores, including continuous contact between the patient and the bedding material that leads to moisture accumulation, lack of air flow to the skin, compression of soft tissues resulting in stagnating blood flow, and inadequate supervision of the patient by care givers.

Many of the aforementioned solutions suffer from one or more drawbacks, such as: mechanical complexity leading to high cost or frequent break-down and maintenance, a need for constant supervision by the care provider to detect the presence or development of bed sores, undesirable restraint of a patient's free movement, or ineffectiveness due to failure to address multiple factors at play. For example, some prior art devices seek to restrain the bed sore patient in certain positions in order to relieve pressure on problem areas. Such a device suffers from the drawbacks that it is a complicated device which must be built into a bed or anchored to it in such a manner as requiring careful expertise, and that is requires constant supervision so that the relief of pressure in one area does not unduly create pressure in another area of the patient's body.

Thus, there remains an urgent need, for a bed sore prevention apparatus that is relatively inexpensive, uncomplicated, and effective. It is especially desired that such a device not unduly restrain a patient's free movement, and addresses both the healing and treatment of existing bed sores and the prevention of new bed sores.

### BRIEF SUMMARY

The present disclosure provides device, optionally for use with a bed, chair, or other furniture for seating, reclining, or lying down, which comprises one or more pressure sensors, a perforated air-flow manifold, a plurality of vibrational motors, and a controller unit, and optionally comprising multiple zones. The device may further comprise one or more temperature sensors. In some embodiments, the device comprises two zones, three zones, four zones, six zones, eight zones, or nine zones. Each zone may be independently monitored and controlled by the control unit. Each zone may have at least one vibrational motor and at least one pressure sensor.

The device of the invention is useful for the treatment and care of any patients who spend a significant of time bed-ridden. The device is useful to help prevent the exacerbation or irritation of pre-existing ulcers (e.g., bed sores), to prevent the formation of new ulcers (e.g., bed sores), and to promote the healing of pre-existing ulcers (e.g., bed sores).

The control unit serves to monitor the input from the one or more pressure sensors, and optionally the one or more temperature sensors. When a pre-determined threshold of pressure or temperature is exceeded, the control unit will automatically initiate a corrective action, e.g., the initiation of vibration, air flow, or both, within those zones of the device which exceeded the pressure or temperature threshold. In some embodiments, the pressure and/or temperature thresholds are freely adjustable by the healthcare provider and/or patient by use of an interface on the control unit (e.g., an alpha-numeric display or flat-screen display with a keypad or keyboard, or a laptop or desktop computer).

In one embodiment, the device further comprises a uniform layer of pressure-re-distributing material, e.g., a foam or gel.

In some embodiments, the device of the invention is an overlay, e.g., a furniture overlay or mattress overlay, that can be laid over and/or secured to any common mattress or other furniture in order to provide the benefits of the invention. In other embodiments, the device of the invention is a complete mattress in which the device of the invention is integrated into the upper (patient-surface) of the mattress.

In another aspect, the present disclosure provides a method of preventing or treating bed sores comprising having a patient in need thereof sleep on a bed comprising a multi-zone, pressure-sensing device, which device comprises one or more pressure sensors, a perforated air-flow manifold, a plurality of vibrational motors, and a controller unit (e.g., a bed with the device integrated into the mattress, or with the device applied to the mattress as a mattress overlay).

In another aspect, the present disclosure provides use of a bed, chair, or other furniture for seating, reclining, or lying down, comprising a pressure-sensing device, which device comprises one or more pressure sensors, a perforated air-flow manifold, a plurality of vibrational motors, and a controller unit (e.g., a bed with the device integrated into the mattress, or with the device applied to the mattress as a mattress overlay) for the prevention or treatment of bed sores, e.g., in a patient in need thereof. Optionally, the device utilizes a multi-zone design (e.g., where each defined zone comprises independently operated motors, sensors and/or air flow elements).

In another aspect, the present disclosure provides use of a pressure-sensing device, which device comprises one or more pressure sensors, a perforated air-flow manifold, a plurality of vibrational motors, and a controller unit for the manufacture of a medical device for the prevention or treatment of bed sores, e.g., in a patient in need thereof (e.g., wherein the medical device is a bed, chair, or other furniture for seating, reclining, or lying down, with the pressure sensing device integrated into the furniture or mattress, or with the device applied to the furniture or mattress as an overlay).

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1. Tri-layer construction of an embodiment of Device 1, showing the upper vibrational motor layer (1), the middle perforated air-flow manifold layer (2), and the lower pressure redistribution layer (3).

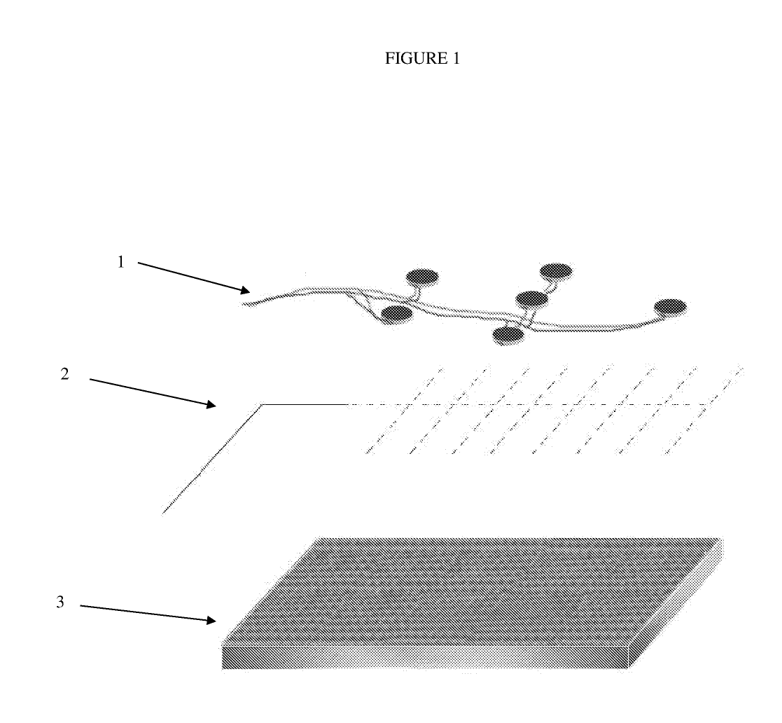

FIG. 2. Exemplary layout of vibrational motors and pressure sensors in a two-zone configuration, showing vibrational motors (1), pressure sensors (2), and zones (3) and (4). Also shown is an exemplary layout of a perforated air-flow manifold tubing (5) with airflow inlet indicated by block arrow.

### DETAILED DESCRIPTION

The following description of the preferred embodiment(s) is merely exemplary in nature and is in no way intended to limit the invention, its application, or uses.

The present disclosure provides device (Device 1) for use with a bed, chair, or other furniture for seating, reclining, or lying down, which comprises one or more pressure sensors, a perforated air-flow manifold, a plurality of vibrational motors, and a controller unit. Optionally, the device comprises one or more zones. The device may further comprise one or more temperature sensors. In some embodiments, each zone is independently monitored and controlled by the control unit. Each zone has at least one vibrational motor and at least one pressure sensor.

In certain embodiments, the present disclosure provides:
1.1 Device 1, wherein the device comprises two zones, three zones, four zones, six zones, eight zones, or nine zones.
1.2 Device 1, or 1.1, wherein the device comprises from one to ten vibrational motors in each zone, e.g., two to eight vibrational motors, two to six vibrational motors, three to six vibrational motors, four to six vibrational motors, or, e.g., six vibrational motors, in each zone.
1.3 Any foregoing device, wherein the vibrational motors are shaftless DC motors, e.g., sized from 6-12 mm or 4-8 mm, optionally, wherein the motors are precision haptic motors, further optionally, wherein the motors operate at 1.5-6V, e.g., 1.5-3V.
1.4 Any foregoing device, wherein the vibrational motors provide a frequency of vibration from 20 to 120 Hz, for example, 20-100 Hz, or 80-120 Hz, or 40-120 Hz, or 40-100 Hz, or 40-60 Hz, or 60-100 Hz, or 60-80 Hz, or, for example, about 47 Hz, about 50 Hz, about 60 Hz, about 70 Hz, about 80 Hz, about 90 Hz or about 100 Hz.
1.5 Any foregoing device, wherein the device comprises from one to ten pressure sensors in each zone, e.g., two to eight pressure sensors, two to six pressure sensors, three to six pressure sensors, four to six pressure sensors, or, e.g., six pressure sensors, in each zone.
1.6 Any foregoing device, wherein the pressure sensors are positioned for detection of pressure in body regions prone to ulcer formation, e.g., one or more of the elbow, inner knees, rear of the head, back of the shoulders, lower back, hip, greater trochanter, and heel.
1.7 Any foregoing device, wherein each vibrational motor is activated when instructed by the control unit to provide vibration to a specific zone.
1.8 Any foregoing device, wherein activation of any particular vibrational motor is based on the detection of above-threshold pressure in closest adjacent pressure sensors.
1.9 Any foregoing device, wherein the pressure sensors are force-sensitive resistors, e.g., square force-sensitive resistors.
1.10 Any foregoing device, wherein the vibrational motors and pressure sensors are secured to the same side of a fabric sheet (e.g., a wool sheet), optionally positioned to minimize interference between and among the motors and sensors.
1.11 Any foregoing device, further comprising one or more temperature sensors.
1.12 Any foregoing device, wherein the device comprises one temperature sensor or two temperature sensors, or from one to ten temperature sensors in each zone, e.g., two to eight temperature sensors, two to six temperature sensors, three to six temperature sensors, four to six temperature sensors, or, e.g., six temperature sensors, in each zone.
1.13 Any foregoing device, wherein each temperature sensor is integrated with each pressure sensor and/or with the vibrational motor.
1.14 Any foregoing device, wherein the perforated airflow manifold is connected to a source of compressed air (e.g., a compressor integrated with the controller, or, e.g., an external source of compressed air), optionally, wherein the pressure of the air provided is controllable (e.g., controllable by an external means, or controllable by the control unit).
1.15 Any foregoing device, wherein the perforated airflow manifold provides airflow to one or more zones of the device when instructed by the control unit.
1.16 Any foregoing device, wherein the perforated airflow manifold comprises one or more control valves to direct air flow to one or more zones of the device.
1.17 Device 1.15 or 1.16, wherein the perforated airflow manifold provides controlled airflow to one more zones of the device by activating one or more of the control valves, optionally, where the control unit controls the air pressure at the input of the airflow manifold to provide a designated pressure at the exit perforations of the manifold (which pressure depends on how many zones airflow is being provided to).
1.18 Any foregoing device, wherein the detection of a pressure exceeding a threshold value (e.g., 35 mm Hg) in a particular zone will result in the control unit actuating the vibrational motors and/or air flow to said zone, optionally wherein said threshold pressure is determined by the user (e.g., the patient or health care provider).
1.19 Any foregoing device, wherein the detection of a temperature exceeding a threshold value in a particular zone will result in the control unit actuating the vibrational motors and/or air flow to said zone, optionally wherein said threshold temperature is determined by the user (e.g., the patient or health care provider).
1.20 Any foregoing device, wherein the detection of a temperature exceeding a maximum allowable threshold value in a particular zone will result in the control unit deactivating the vibrational motors in that zone, e.g., to reduce the risk of overheating of the motors and/or the risk of fire.
1.21 Any foregoing device, wherein the control unit measures pressure and/or temperature at each sensor at a predetermined schedule (e.g., every one minute, two minutes, three minutes, four minutes, five minutes, ten minutes, fifteen minutes, twenty minutes, etc.), optionally, wherein the control unit records each such measurement in a storage medium (e.g., a hard drive, floppy disk, flash drive, flash memory, etc.), and optionally, wherein the control unit can recall and compare previous pressure and temperature records to current measurements.
1.22 Any foregoing device, wherein the control unit shuts off the vibrational motors and/or air flow to a particular zone after a pre-determined running time (e.g., one minute, two minutes, three minutes, four minutes, five minutes, ten minutes, fifteen minutes, twenty minutes, etc.), optionally wherein the pre-determined running time is selected to prevent or avoid overheating of the motors.
1.23 Any foregoing device, wherein the control unit shuts off the vibrational motors and/or air flow to a particular zone when the pressure and/or temperature sensors in that zone have detected that the pressure and/or temperature has fallen below a preset threshold value, optionally wherein said threshold value(s) is/are determined by the user (e.g., the patient or health care provider).
1.24 Any foregoing device, wherein the control unit comprises an alpha-numeric keyboard or keypad for the entry of patient information and/or temperature or pressure thresholds (e.g., a pressure threshold of 35 mm Hg, and/or a temperature threshold of 75° F.).
1.25 Any foregoing device, wherein the control unit is connected to a laptop computer, desktop computer, tablet computer, smart phone or other commercial or consumer electronic device as a user interface.
1.26 Any foregoing device, wherein the control unit can be connected to a central electronic patient management system to enable remote control or monitoring of the device (e.g., at a hospital nurse or doctor's station).
1.27 Any foregoing device, wherein the device further comprises a uniform layer of pressure-re-distributing material, e.g., a foam or gel, for example, a viscoelastic foam or gel.
1.28 Device 1.27, wherein the foam or gel is a microporous, gel for example, an optionally cross-linked polyester, polyamide, polystyrene, polyurethane, polyether, polyvinyl or polyvinylidene polymer or copolymer.
1.29 Any foregoing device, wherein the device comprises one or more layers, wherein at least one layer contains the vibrational motors, and another layer contains the pressure sensors, and another layer contains the airflow manifold.
1.30 Device 1.29, wherein the pressure sensors and vibrational motors are contained in the same layer.
1.31 Device 1.29 or 1.30, wherein the airflow manifold layer is below the layer or layers containing the pressure sensors and vibrational motors.
1.32 Any of Devices 1.29-1.31, wherein the pressure re-distribution layer (e.g., the foam or gel layer) is below the layers containing the vibrational motors and/or pressure sensors, for example, wherein the pressure re-distribution layer is the bottom layer.
1.33 Any of Devices 1.29-1.32, wherein the vibrational motors and pressure sensors are in a top layer, the airflow manifold is in a middle layer, and the pressure re-distribution layer is the bottom layer.
1.34 Any of Devices 1.29-1.33 where one or more of the layers are separated by a fabric sheet, optionally, where each layer is separated by a fabric sheet (e.g., a wool, cotton, polyester or elastic sheet), optionally a wool sheet.
1.35 Any foregoing device, wherein any and all vibrational motors, pressure sensors, temperature sensors, airflow manifold and fabric inter-layer sheets are contained in a fabric enclosure for application to a bed.
1.36 Any foregoing device, wherein the device is a mattress overlay, e.g., a bed sheet, for removable use on a standard bed mattress
1.37 Any of Devices 1-1.34, wherein the device is integrated into the upper layer of a mattress.
1.38 Any foregoing device, wherein the control unit is in a separate housing from the operating elements of the device (e.g., the vibrational motors, pressure sensors, temperature sensors, airflow manifold and fabric inter-layer sheets).

The control unit of the device serves to monitor the input from the one or more pressure sensors, and optionally the one or more temperature sensors. The control unit optionally comprises a computer processor, e.g., a microprocessor or integrated circuit, configured to provide monitoring, display and control functions. For example, when a pre-determined threshold of pressure or temperature is exceeded, the control unit will automatically initiate a corrective action, e.g., the initiation of vibration, air flow, or both, within those zones of the device which exceeded the pressure or temperature threshold. In some embodiments, the pressure and/or temperature thresholds are freely adjustable by the healthcare provider and/or patient by use of an interface on the control unit (e.g., an alpha-numeric display or flat-screen display with a keypad or keyboard, or a laptop or desktop computer). In some embodiments the control unit is an independent electronic device, whereas in other embodiments, the control unit is an interface which is controlled and operated by a computer. In the latter case, the computer may be supplied with a software program or algorithm which provides the instructions necessary to operate the control unit.

Without being bound by theory, it is believed that increased pressure between a patient's body and a patient's bedding is associated with a higher risk of bed sore development. The pressure exerted results in the compression of soft tissues, the inhibition of capillary blood flow, and the accumulation of moisture between the patient and the bedding. Without being bound by theory, it is believed that activation of the vibratory motors and/or activation of air flow in the region of the increased pressure serves to reduce that risk of bed sore development. The vibratory motors cause a vibration of the soft tissue under pressure which results in increased capillary blood flow, while the air flow helps evaporate the accumulated moisture from both the bedding material and the patient's clothing and skin. As a result, the device of the present disclosure helps ameliorate the two key factors which connect increased pressure to bed sore development. In today's busy health care climate, this would help prevent the development of bed sores in immobile or poorly mobile patients in between actions taken by a health care provider to directly relieve bed pressure (e.g., by turning or rotating a patient, or by providing for ambulation).

In one embodiment, the device further comprises a uniform layer of pressure-re-distributing material, e.g., a foam or gel. Without out being bound by theory, it is believed that a layer of foam or gel of sufficient thickness and adequate density and resilience will help redistribute a patient's body weight to help ensure a uniform distribution of pressure across the patient's body. In some embodiments, the pressure-redistributing material also enhances the efficacy of the vibrational motors by extending the distance over which the vibration is effective, as well as reducing local irritation in the vicinity of the motor by spreading out the impact of the motors' vibration. In addition, the use of a pressure redistribution layer helps further prevent the exertion of pressure on soft tissues than can cause a restriction in local cutaneous or subcutaneous blood flow (which can lead to tissue damage, including tissue necrosis). These factors may further reduce the risk of bed sore formation and enhance bed sore healing. Studies have shown that the a suitable pressure to be maintained on the body to prevent the development of bed sores is from 10 to 44 mm Hg, for example, from 15 to 35 mm Hg, or from 16 to 33 mm Hg.

In some embodiments, the vibrational motors operate at a frequency of from 20 to 120 Hz, for example, 20-100 Hz, or 80-120 Hz, or 40-120 Hz, or 40-100 Hz, or 40-60 Hz, or 60-100 Hz, or 60-80 Hz, or, for example, about 47 Hz, about 50 Hz, about 60 Hz, about 70 Hz, about 80 Hz, about 90 Hz or about 100 Hz. Without being bound by theory, it is believed that certain operational frequencies for the vibration results in better improvements in capillary blood flow. In particular embodiments, the preferred frequency is about 47 Hz. In other particular embodiments, the preferred frequency is about 100 Hz.

In preferred embodiments, the vibrational motors are positioned at least 5 inches from each other in order to reduce or eliminate interference between them, for example, constructive and destructive interference. In some embodiments, the motors are positioned at a distance apart from each sufficient to prevent interference, for example, from 5 to 15 inches, or 5 to 10 inches, or 5 to 7 inches, or about 5 inches, about 6 inches, about 7 inches, or about 6.5 inches. In preferred embodiments, the vibrational motors are also positioned at least 3 inches apart from any pressure sensors to avoid or reduce interference between them (for example 3-15 inches, or 3-10 inches, or 4-10 inches, or 4-8 inches, or 4-6 inches, or 4-5 inches apart). In preferred embodiments, the pressure sensors are positions at least 5 inches apart from each other, to reduce or prevent interference between them (for example, 5-15 inches, or 5-10 inches, or 6-8 inches, or about 7 inches apart). In some embodiments the motors are arranged so as to be distributed evenly or substantially evenly over the area of the device.

In some embodiments, the invention further comprises one or more temperature sensors. The temperature sensors can operate to serve either of two purposes, or both purposes. First, the temperature sensors, when multiple sensors are used, can serve to relay to the control unit information about the patient's body temperature which, in combination with the pressure sensor information, can help identify regions of the patient at risk for the development of bed sores (e.g., a higher temperature may indicated a region of compressed soft tissue or infected soft tissue). Second, the temperature sensors, even when only a single sensor is used, can be used to guard against over-heating of the device resulting from operation of the vibrational motors. For example, either due to long duration of operation or due to electrical or mechanical fault, one or vibrational motors can overheat. Such overheating presents the risks of thermal injury to the patient, thermal damage to the device or the mattress underneath the device, and a risk of fire in the bed sheets or other materials in the vicinity. The use of one or more temperature sensors to monitor the temperature can be used a safety mechanism to automatically shut off the motors if a threshold dangerous temperature is exceeded. In addition, the control unit can be configured to increase the flow of air from the airflow manifold in order to reduce the temperature of the patient, or the vibrational motors, or the bedding material, or some combination thereof.

In some embodiments, the control unit includes an air compressor, and optionally, one or more air tanks, for the purpose of providing compressed air to the airflow manifold. In other embodiments, the control unit further contains one or more pressure regulating devices for control of the air pressure provided to the airflow manifold. Such pressure regulating devices may be controlled via the control unit in order to provide an optimum flow rate (e.g., mass or volume rate) of air to the manifold.

In other embodiments, the control unit may include a port for the attachment of an external supply of compressed air. In some embodiments, the control unit includes both an air compressor, optionally with one or more air tanks, and a port for the attachment of an external supply of compressed air, such that the device can be operating using either internal or external compressed air.

In some embodiments, the device of the invention is an overlay, e.g., a mattress overlay that can be laid over and/or secured to any common mattress in order to provide the benefits of the invention. In other embodiments, the device of the invention is a complete mattress in which the device of the invention is integrated into the upper (patient-surface) of the mattress.

In another aspect, the present disclosure provides a method of preventing or treating bed sores comprising having a patient in need thereof sleep on a bed comprising a multi-zone, pressure-sensing device, which device comprises one or more pressure sensors, a perforated air-flow manifold, a plurality of vibrational motors, and a controller unit (e.g., a bed with the device integrated into the mattress, or with the device applied to the mattress as a mattress overlay).

In a particular embodiment, the device used in the above described method, in any of its embodiments, is a device of the present disclosure, e.g., Device 1 or any of Devices 1.1-1.38.

In another aspect, the present disclosure provides use of a bed comprising a multi-zone, pressure-sensing device, which device comprises one or more pressure sensors, a perforated air-flow manifold, a plurality of vibrational motors, and a controller unit (e.g., a bed with the device integrated into the mattress, or with the device applied to the mattress as a mattress overlay) for the prevention or treatment of bed sores, e.g., in a patient in need thereof.

In another aspect, the present disclosure provides use of a multi-zone, pressure-sensing device, which device comprises one or more pressure sensors, a perforated air-flow manifold, a plurality of vibrational motors, and a controller unit for the manufacture of a medical device for the prevention or treatment of bed sores, e.g., in a patient in need thereof (e.g., wherein the medical device is a bed with the multi-zone, pressure sensing device integrated into the mattress, or with the device applied to the mattress as a mattress overlay). In a particular embodiment, the device used is a device of the present disclosure, e.g., Device 1 or any of Devices 1.1-1.38.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

The invention of the present disclosure will become clearer with reference to the following Examples. The Examples are not intended in any way to limit the scope of the invention disclose and merely serve as exemplary embodiments of the invention.

### EXAMPLE 1

### Exemplary Design of Smart Sheet Mattress Overlay

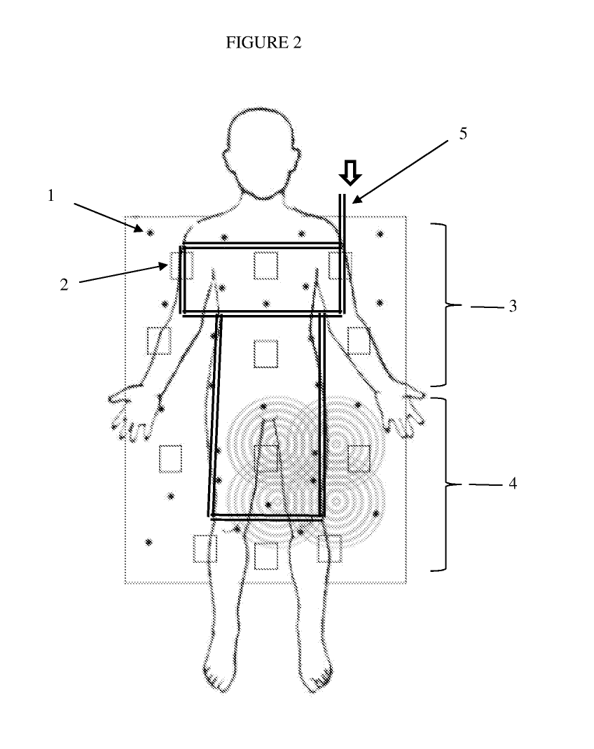

A two-zone mattress overlay is constructed using 27 vibrational motors, 12 pressure sensors and one temperature sensor. FIG. 2 shows the arrangement of the motors and sensors, which lie in a single layer between sheets of fabric. In a second layer, below this first layer, lies the perforated airflow manifold, which is designed as a double-loop arrangement. This arrangement provides effect airflow to all areas of a patient's body. The entire mattress overlay is enclosed in a fabric container which is easily moved from one bed to another, and can also be rolled up for easy storage when not in use. The vibrational motors are medical grade Precision Haptic DC motors operating at 100 Hz frequency.

Attached via cabling and compressed air hosing to the mattress overlay is the control unit. The control unit is centered on an Arduino Mega 2560 microprocessor, which controls all aspects of the device's operation. The user interface is provided by connection to a standard, commercially available laptop computer.

### EXAMPLE 2

### Effect of Underlying pressure-redistibuting gel layer

The mattress overlay of Example 1 is operated with a human patient lying in a supine position either with or without an air-permeable, hydrophobic gel layer six-inches in thickness underneath the mattress overlay. The pressure exerted by the patient on the mattress overlay is recorded by nine pressure sensors over a period of about 200 seconds. It is found that in the absence of the gel layer, only 8 of the pressure sensors show a force exerted of under 5 Newtons. One sensor, however, consistently shows a force exerted of from 10 to 45 Newtons. This is considerably higher than is desirable, and would suggest a higher risk of bed sore development for a patient lying supine on such a mattress. In contrast, when the mattress overlay is used on top of the hydrophobic gel, sensor 8 shows a consistent force exerted of only about 8 Newtons, while the remaining sensors all register below 5 Newtons. This demonstrates that the gel layer helps redistribute pressure more uniformly throughout the mattress overlay, which will provide for improved healing of existing bed sores, as well as the preventing of new bed sores.

### EXAMPLE 3

### Effect of Airflow Manifold

The design of Example 1 utilizes a double-rectangular loop airflow manifold as shown in FIG. 2. Perforations are placed around the length of the tubing (diameter ¼ inch) approximately every five inches. Compressed air is supplied at a pressure of 60 psi, and the airflow was turned on for two-minute intervals every sixteen minutes, while temperature was recorded at five second intervals. Temperature was measured at the single sensor under the center of the patient's body. The patient did not substantially move for the duration of the 30 minute test. The results demonstrate that absent airflow, the temperature sensor gradually recorded higher temperature as heat and moisture accumulated between the patient's body and the mattress overlay. In contrast, with the intermittent running of the airflow manifold, the temperature sensor's recorded temperature remained approximately constant.

What is claimed is:

1. A device for use with a bed, chair, or other furniture for seating, reclining, or lying down, which comprises one or more pressure sensors, a perforated air-flow manifold, a plurality of vibrational motors, and a controller unit.

2. The device of claim 1, wherein the device comprises one or more zones, for example, two zones, three zones, four zones, six zones, eight zones, or nine zones.

3. The device of claim 1 or 2, wherein the device comprises from one to ten vibrational motors in each zone, e.g., two to eight vibrational motors, two to six vibrational motors, three to six vibrational motors, four to six vibrational motors, or, e.g., six vibrational motors, in each zone.

4. Any foregoing device, wherein the device comprises from one to ten pressure sensors in each zone, e.g., two to eight pressure sensors, two to six pressure sensors, three to six pressure sensors, four to six pressure sensors, or, e.g., six pressure sensors, in each zone.

5. Any foregoing device, wherein the pressure sensors are positioned for detection of pressure in body regions prone to ulcer formation, e.g., one or more of the elbow, inner knees, rear of the head, back of the shoulders, lower back, hip, greater trochanter, and heel.

6. Any foregoing device, further comprising one or more temperature sensors.

7. Any foregoing device, wherein the perforated airflow manifold is connected to a source of compressed air (e.g., a compressor integrated with the controller, or, e.g., an external source of compressed air), optionally, wherein the pressure of the air provided is controllable (e.g., controllable by an external means, or controllable by the control unit).

8. Any foregoing device, wherein the perforated airflow manifold provides airflow to one or more zones of the device when instructed by the control unit.

9. Any foregoing device, wherein the detection of a pressure exceeding a threshold value in a particular zone will result in the control unit actuating the vibrational motors and/or air flow to said zone, optionally wherein said threshold pressure is determined by the user (e.g., the patient or health care provider).

10. Any foregoing device, wherein the detection of a temperature exceeding a threshold value in a particular zone will result in the control unit actuating the vibrational motors and/or air flow to said zone, optionally wherein said threshold temperature is determined by the user (e.g., the patient or health care provider).

11. Any foregoing device, wherein the control unit measures pressure and/or temperature at each sensor at a predetermined schedule (e.g., every one minute, two minutes, three minutes, four minutes, five minutes, ten minutes, fifteen minutes, twenty minutes, etc.), optionally, wherein the control unit records each such measurement in a storage medium (e.g., a hard drive, floppy disk, flash drive, flash memory, etc.), and optionally, wherein the control unit can recall and compare previous pressure and temperature records to current measurements.

12. Any foregoing device, wherein the control unit shuts off the vibrational motors and/or air flow to a particular zone after a pre-determined running time (e.g., one minute, two minutes, three minutes, four minutes, five minutes, ten minutes, fifteen minutes, twenty minutes, etc.), optionally wherein the pre-determined running time is selected to prevent or avoid overheating of the motors.

13. Any foregoing device, wherein the control unit shuts off the vibrational motors and /or air flow to a particular zone when the pressure and/or temperature sensors in that zone have detected that the pressure and/or temperature has fallen below a preset threshold value, optionally wherein said threshold value(s) is/are determined by the user (e.g., the patient or health care provider).

14. Any foregoing device, wherein the device further comprises a uniform layer of pressure-re-distributing material, e.g., a foam or gel, for example, a viscoelastic foam or gel.

15. The device of claim 14, wherein the foam or gel is a microporous, gel for example, an optionally cross-linked polyester, polyamide, polystyrene, polyurethane, polyether, polyvinyl or polyvinylidene polymer or copolymer.

16. Any foregoing device, wherein the device comprises one or more layers, wherein at least one layer contains the vibrational motors, and another layer contains the pressure sensors, and another layer contains the airflow manifold.

17. The device of claim 16, wherein the pressure sensors and vibrational motors are contained in the same layer, optionally, wherein the airflow manifold layer is below the layer or layers containing the pressure sensors and vibrational motors, further optionally wherein the pressure re-distribution layer (e.g., the foam or gel layer) is below the layers containing the vibrational motors and/or pressure sensors, for example, wherein the pressure re-distribution layer is the bottom layer.

18. A method of preventing or treating bed sores comprising having a patient in need thereof sleep on a bed comprising a multi-zone, pressure-sensing device according to any one of claims 1 to 17.

19. Use of a device according to any of claims 1 to 17 for the prevention or treatment of bedsores in a patient in need thereof.
